(19) United States
(12) Patent Application Publication
(10) Pub. No.: US 2019/0187278 A1
(43) Pub. Date: Jun. 20, 2019

(54) ULTRASOUND PROBE AND ULTRASOUND DIAGNOSTIC APPARATUS

(71) Applicant: Konica Minolta, Inc., Tokyo (JP)

(72) Inventors: Masashi OZAWA, Yokohama-shi (JP); Yuiko KITAMURA, Tokyo (JP); Kaoru OKADA, Tokyo (JP)

(21) Appl. No.: 16/181,425

(22) Filed: Nov. 6, 2018

(30) Foreign Application Priority Data
Dec. 20, 2017 (JP) 2017-243897

(51) Int. Cl. G01S 15/89; A61B 8/00; G10K 11/02; G01S 7/52

(52) U.S. Cl. CPC G01S 15/8915; A61B 8/4483; H03H 7/38; G01S 7/5208; G10K 11/02

(57) ABSTRACT

An ultrasound probe of an ultrasound diagnostic apparatus, includes: a plurality of ultrasound transducers that perform mutual conversion between an ultrasound wave and an electrical signal; a switching circuit that selectively switches ultrasound transducers to be electrically connected to a transmitting/receiving circuit among the plurality of ultrasound transducers; and a matching circuit that is provided at one of a preceding stage and a subsequent stage of the switching circuit, is connected to a position connected directly to the switching circuit, and performs impedance matching between the switching circuit and a circuit disposed at one of the preceding stage and the subsequent stage of the switching circuit.

The entire disclosure of Japanese patent Application No. 2017-243897, filed on Dec. 20, 2017, is incorporated herein by reference in its entirety.

### BACKGROUND

### Technological Field

The present disclosure relates to an ultrasound probe and an ultrasound diagnostic apparatus.

### Description of the Related Art

A conventional ultrasound diagnostic apparatus has ultrasound transducers disposed in an ultrasound probe.

In an ultrasound diagnostic apparatus of this type, the main body of the ultrasound diagnostic apparatus and the ultrasound probe are normally connected by a cable, and the number of ultrasound transducers connected to the main body is restricted by the number of signal lines in the cable and the number of system channels of a transmitting/receiving circuit.

In an ultrasound diagnostic apparatus of this type, the ultrasound transducers to be driven are switched and controlled in a time-sharing manner by switching circuits called multiplexers, so that transmission/reception beam deflection control and transmission/reception beam aperture movement are achieved (see JP 2006-288547 A, for example).

As for such an ultrasound diagnostic apparatus, there is a demand for higher target-detection sensitivity, higher ultrasound image resolution, and the like (which will be hereinafter referred to as the “acoustic characteristics of ultrasound transducers”). In view of this, reducing the signal degradation between the ultrasound transducers and the transmitting/receiving circuit is an important goal.

As a result of intensive studies, the inventors of this application have arrived at the conclusion that the above switching circuits (multiplexers, for example) are the cause of the signal degradation. Specifically, a switching circuit has a different circuit constant from that of the signal line extending from the transmitting/receiving circuit side. Therefore, when a transmission signal is transmitted from the transmitting/receiving circuit to an ultrasound transducer, or when a reception signal is transmitted from an ultrasound transducer to the transmitting/receiving circuit, a signal reflection phenomenon or the like might be caused, and the reflection phenomenon might cause signal degradation.

JP 2006-288547 A teaches that there is a difference in signal strength between a circuit including a switching circuit and a circuit not including any switching circuit, and, in view of this, a resistor equivalent to the on-resistance of a switching circuit is inserted into a circuit not including any switching circuit. However, JP 2006-288547 A does not mention any signal reflection phenomenon in switching circuits, and is unable to solve signal degradation caused by such a reflection phenomenon.

### SUMMARY

The present disclosure has been made in view of the above problems, and an object thereof is to provide an ultrasound probe and an ultrasound diagnostic apparatus that are capable of reducing signal degradation caused by a switching circuit that selectively switches targets to be driven among a plurality of ultrasound transducers.

To achieve the abovementioned object, according to an aspect of the present invention, an ultrasound probe of an ultrasound diagnostic apparatus reflecting one aspect of the present invention comprises:

a plurality of ultrasound transducers that perform mutual conversion between an ultrasound wave and an electrical signal;

a switching circuit that selectively switches ultrasound transducers to be electrically connected to a transmitting/receiving circuit among the plurality of ultrasound transducers; and

a matching circuit that is provided at one of a preceding stage and a subsequent stage of the switching circuit, is connected to a position connected directly to the switching circuit, and performs impedance matching between the switching circuit and a circuit disposed at one of the preceding stage and the subsequent stage of the switching circuit.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram showing the exterior of an ultrasound diagnostic apparatus according to a first embodiment;

FIG. 2 is a block diagram showing the configuration of the entire ultrasound diagnostic apparatus according to the first embodiment;

FIG. 3 is a circuit diagram showing the configuration of an ultrasound probe according to the first embodiment;

FIGS. 4A and 4B are diagrams showing the array structure of ultrasound transducers according to the first embodiment;

FIGS. 5A and 5B are diagrams for explaining switching operations at multiplexers according to the first embodiment;

FIGS. 6A and 6B are diagrams showing equivalent circuits of multiplexers;

FIG. 7 shows the results of a simulation conducted to determine the transmission/reception characteristics of the ultrasound probe according to the first embodiment;

FIGS. 8A and 8B are diagrams showing a mounting structure for the circuit components of the ultrasound probe according to the first embodiment;

FIGS. 9A and 9B are diagrams showing a modification of the mounting structure for the circuit components of the ultrasound probe; and

FIG. 10 is a circuit diagram showing the configuration of an ultrasound probe according to a second embodiment.

### DETAILED DESCRIPTION OF EMBODIMENTS

Hereinafter, one or more preferred embodiments of the present disclosure will be described in detail with reference to the drawings. However, the scope of the invention is not limited to the disclosed embodiments. In this specification and the drawings, components having substantially the same function are denoted by the same reference numeral so that the same explanation will not be repeated.

### First Embodiment

[Configuration of an Entire Ultrasound Diagnostic Apparatus]

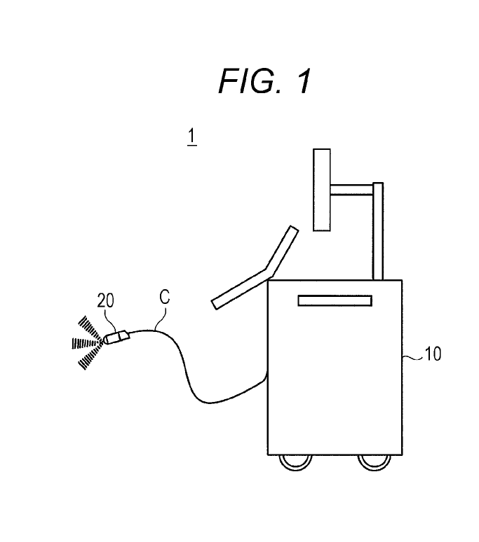

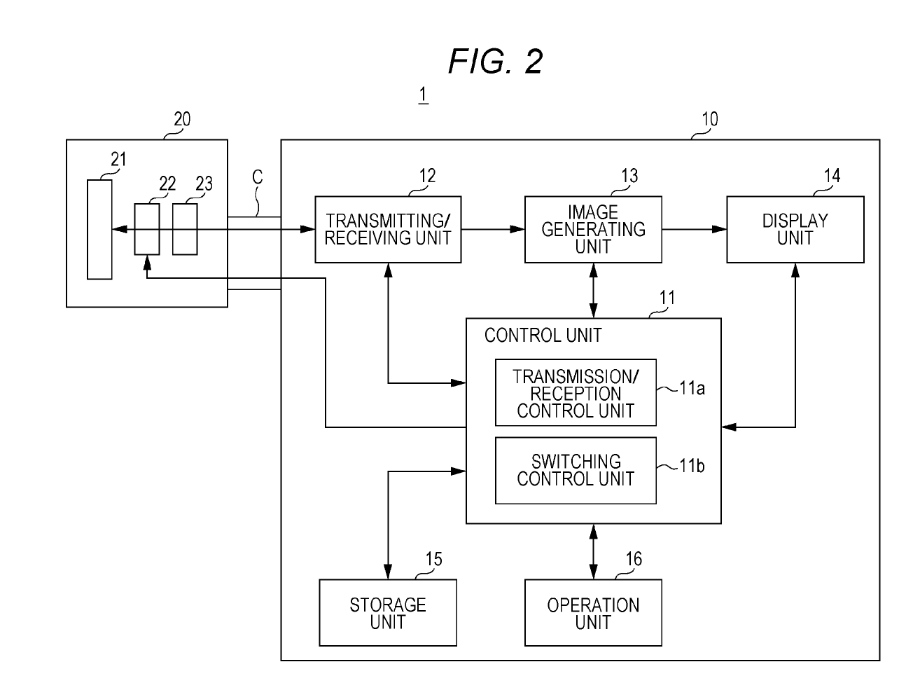

Referring to FIGS. 1 and 2, an example of the configuration of an entire ultrasound diagnostic apparatus 1 according to this embodiment is described below.

FIG. 1 is a diagram showing the exterior of the ultrasound diagnostic apparatus 1 according to this embodiment. FIG. 2 is a block diagram showing the configuration of the entire ultrasound diagnostic apparatus 1 according to this embodiment.

The ultrasound diagnostic apparatus 1 according to this embodiment has an ultrasound probe 20 attached to a main body 10 of the ultrasound diagnostic apparatus 1 (this main body will be hereinafter referred to simply as the “main body 10”). The main body 10 and the ultrasound probe 20 are electrically connected via a cable C.

Note that the ultrasound diagnostic apparatus 1 according to this embodiment may generate any ultrasound image, such as a B-mode image, a color Doppler image, a three-dimensional ultrasound image, or an M-mode image. Likewise, any ultrasound probe, such as a convex probe, a linear probe, a sector probe, or a three-dimensional probe, can be used as the ultrasound probe 20.

The main body 10 of the ultrasound diagnostic apparatus 1 includes a control unit 11, a transmitting/receiving unit 12, an image generating unit 13, a display unit 14, a storage unit 15, and an operation unit 16. Meanwhile, the ultrasound probe 20 includes ultrasound transducers 21, switching circuits 22, and matching circuits 23.

The transmitting/receiving unit 12 (hereinafter also referred to as the “transmitting/receiving circuit 12”) is a transmitting/receiving circuit that causes the ultrasound transducers 21 of the ultrasound probe 20 to transmit and receive ultrasound waves. The transmitting/receiving unit 12 includes a transmitting circuit that generates a voltage pulse (hereinafter referred to as a “transmission signal”) and transmits the voltage pulse to the ultrasound transducers 21, and a receiving circuit that performs a reception process (an amplification process and an A-D conversion process, for example) on an electrical signal (hereinafter referred to as a “reception signal) relating to an ultrasound echo generated by the ultrasound transducers 21. Under the control of the control unit 11, the transmitting circuit and the receiving circuit each perform an operation to cause the ultrasound transducers 21 to transmit and receive ultrasound waves.

It should be noted that the transmitting/receiving unit 12 has system channels, and is capable of operating the ultrasound transducers 21 for each of the system channels.

The transmitting/receiving unit 12 is connected to the ultrasound transducers 21 of the ultrasound probe 20 via the cable C. Electrical signals are exchanged between the transmitting/receiving unit 12 and the ultrasound transducers 21 via signal lines contained in the cable C. In the ultrasound probe 20 according to this embodiment, any circuit configuration for performing reception processes and transmission processes is not provided, and signals having analog waveforms flow through the signal lines in the cable C.

The image generating unit 13 performs predetermined signal processing (logarithmic compression, wave detection, and FFT analysis, for example) on a reception signal acquired from the transmitting/receiving unit 12, to generate an ultrasound image (a B-mode image, a color Doppler image, or a three-dimensional ultrasound image, for example). Since the details of the process to be performed for generating an ultrasound image are well known, explanation of them are not made herein.

The display unit 14 is a liquid crystal display or the like, for example, and displays an ultrasound image generated by the image generating unit 13. The storage unit 15 is a memory such as a hard disk, a ROM, or a RAM, for example, and stores a control program and various kinds of data (various kinds of setting data to be set in the transmitting/receiving unit 12) to be referred to by the control unit 11, image data generated by the image generating unit 13, and the like. The operation unit 16 is a keyboard, a mouse, or the like, and acquires an operation signal that is input by an operator.

The control unit 11 communicates with the respective components (the transmitting/receiving unit 12, the image generating unit 13, the display unit 14, the storage unit 15, and the operation unit 16) of the ultrasound diagnostic apparatus 1, to comprehensively control these components.

The control unit 11 includes a transmission/reception control unit 11a and a switching control unit 11b.

The transmission/reception control unit 11a causes the transmitting/receiving unit 12 to transmit a transmission signal to each ultrasound transducer 21, and causes the transmitting/receiving unit 12 to perform a reception process on a reception signal from each ultrasound transducer 21.

The switching control unit 11b controls the switching circuits 22, to control switching of the ultrasound transducers 21 to be driven among the ultrasound transducers 21. In other words, the switching control unit 11b controls the switching circuits 22, to perform time-sharing switching control on the ultrasound transducers 21 connected to the respective system channels of the transmitting/receiving unit 12, for example. Switching on and off of driven states of the ultrasound transducers 21 may be controlled individually for each ultrasound transducer 21, or may be controlled on a block-by-block basis.

The control unit 11 includes a central processing unit (CPU), a read only memory (ROM), a random access memory (RAM), an input port, an output port, and the like, for example. Each of the functions described above is achieved when the CPU refers to a control program and various kinds of data stored in the ROM and the RAM. However, some or all of the above functions are not necessarily achieved by processing performed by software, and can of course also be achieved with a dedicated hardware circuit or a combination of software and hardware.

[Configuration of the Ultrasound Probe]

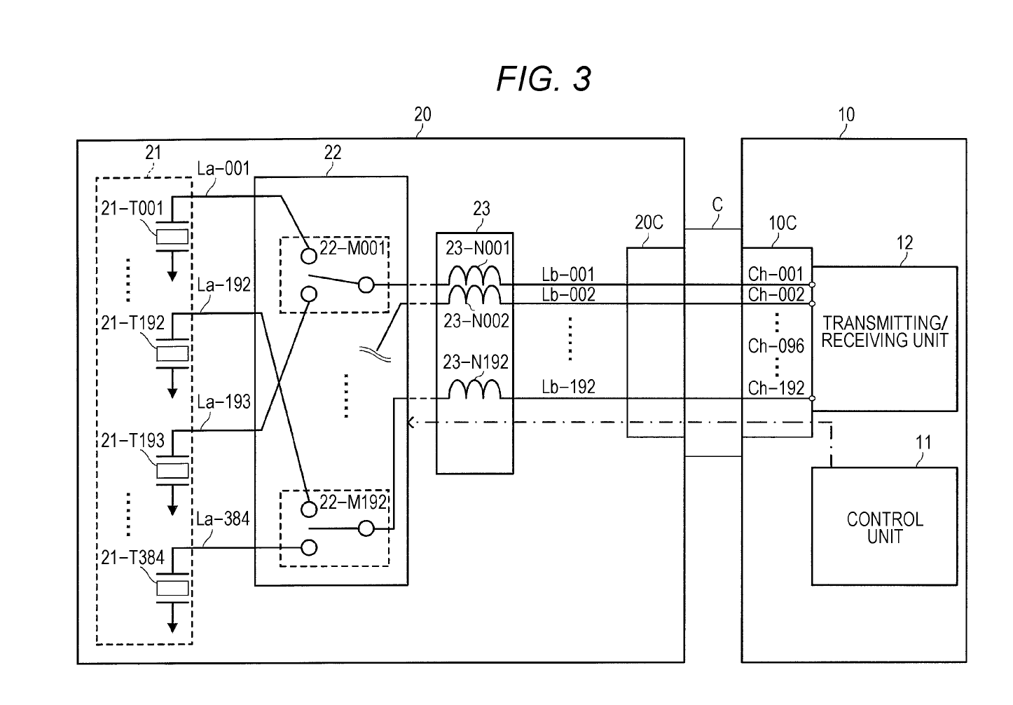

Referring now to FIGS. 3 through 7, an example of the configuration of the ultrasound probe 20 according to this embodiment is described.

FIG. 3 is a circuit diagram showing the configuration of the ultrasound probe 20 according to this embodiment.

The ultrasound probe 20 according to this embodiment includes the ultrasound transducers 21, the switching circuits 22, the matching circuits 23, a cable connector 20C, signal lines La (384 signal lines La-001 through La-384 in this example) on the side of the ultrasound transducers 21, and signal lines Lb (192 signal lines Lb-001 through Lb-192 in this example) on the system channel side.

In the ultrasound probe 20 according to this embodiment, the switching circuits 22, the matching circuits 23, and the cable connector 20C are connected in this order from the side of the ultrasound transducers 21 (hereinafter, the side of the ultrasound transducers 21 will be also referred to as the “preceding-stage side”, and the side of the cable connector 20C will be also referred to as the “subsequent-stage side”).

The transmitting/receiving circuit 12 according to this embodiment has 192 channels (Ch-001 through Ch-192) as the system channels for driving the ultrasound transducers 21. The respective system channels Ch-001 through Ch-192 as the 192 channels are connected to switching circuits 22-M001 through 22-M192 that are different from one another, via the signal lines Lb-001 through Lb-192 connected thereto. The respective system channels Ch-001 through Ch-192 as the 192 channels exchange electrical signals with ultrasound transducers 21-T001 through 21-T384 selected by the switching circuits 22-M001 through 22-M192 connected thereto.

In the ultrasound probe 20 according to this embodiment, the 384 ultrasound transducers 21-T001 through 21-T384, the 192 switching circuits 22-M001 through 22-M192 individually connected to the respective ultrasound transducers 21-T001 through 21-T384, and the 192 matching circuits 23-N001 through 23-N192 individually connected to the respective switching circuits 22-M001 through 22-M192 are arranged adjacent to one another.

The 384 signal lines La-001 through La-384 are disposed between the ultrasound transducers 21-T001 through 21-T384 and the switching circuits 22-M001 through 22-M192, being connected to the respective transducers and the switching circuits. Meanwhile, the 192 signal lines Lb-001 through Lb-192 are disposed between the switching circuits 22-M001 through 22-M192 and the system channels Ch-001 through Ch-192 of the transmitting/receiving circuit 12, being connected to the respective switching circuits and the respective system channels.

The cable C contains the same number of signal lines Lb as the number of system channels (the 192 channels in this example) for driving the ultrasound transducers 21, and control lines (not shown) for performing switching control on the switching circuits 22. Electrical signals are exchanged between the ultrasound probe 20 and the main body 10 via the cable connector 20C on the side of the ultrasound probe 20, the cable C, and a cable connector 10C on the side of the main body 10.

In the description below, the configurations of the respective ultrasound transducers 21-T001 through 21-T384 are assumed to be the same, and are referred to simply as the ultrasound transducers 21 in a case where there is no need to specifically distinguish them from one another. The configurations of the switching circuits 22-M001 through 22-M192 are also assumed to be the same, and are referred to simply as the switching circuits 22 in a case where there is no need to specifically distinguish them from one another. The configurations of the matching circuits 23-N001 through 23-N192 are also assumed to be the same, and are referred to simply as the matching circuits 23 in a case where there is no need to specifically distinguish them from one another. The signal lines La-001 through La-384 are also referred to simply as the signal lines La in a case where there is no need to specifically distinguish them from one another. The signal lines Lb-001 through Lb-192 are referred to simply as the signal lines Lb in a case where there is no need to specifically distinguish them from one another.

Configuration of the Ultrasound Transducers 21

Each ultrasound transducer 21 is a piezoelectric element that performs mutual conversion between ultrasound waves and an electrical signal. The ultrasound transducer 21 converts a transmission signal transmitted from the transmitting/receiving circuit 12 into ultrasound waves, transmits the ultrasound waves into the subject, converts the ultrasound echo reflected within the subject into an electrical signal, and transmits the electrical signal to the transmitting/receiving circuit 12. The ultrasound transducer 21 includes a piezoelectric body such as a piezoceramic material, a polymer piezoelectric material, or a piezoelectric single-crystal material, a signal electrode disposed on one side surface of the piezoelectric body, and a ground electrode disposed on the other side surface of the piezoelectric body, for example. A signal line La is connected to the signal electrode of the ultrasound transducer 21, and a grounding conductor is connected to the ground electrode.

The ultrasound transducers 21 are individually connected to the respective switching circuits 22 via the respective signal lines La, and electrical connection to the transmitting/receiving circuit 12 are selectively switched on and off by switching at the switching circuits 22. Of these ultrasound transducers 21, the ultrasound transducer 21 electrically connected to the transmitting/receiving circuit 12 is the ultrasound transducer 21 to be driven, and exchanges electric signals (a transmission signal and a reception signal) with the transmitting/receiving circuit 12.

In this configuration, under the control of the control unit 11 (the switching control unit 11b), the ultrasound transducer 21 to be driven is selected in a time-sharing manner from among the ultrasound transducers 21, and transmission/reception beam deflection control and electronic scanning such as aperture movement of the transmission/reception beam are performed.

The switching on and off of the electrically connected state (that is, switching between a driven state and a non-driven state) between the ultrasound transducers 21 and the transmitting/receiving circuit 12 may be individually controlled for each ultrasound transducer 21, but may also be controlled for each block of the ultrasound transducers 21. Alternatively, switching control may be performed so that each ultrasound transducer 21 that performs an ultrasound wave transmitting operation and each ultrasound transducer 21 that performs an ultrasound echo receiving operation are separated from each other among the ultrasound transducers 21.

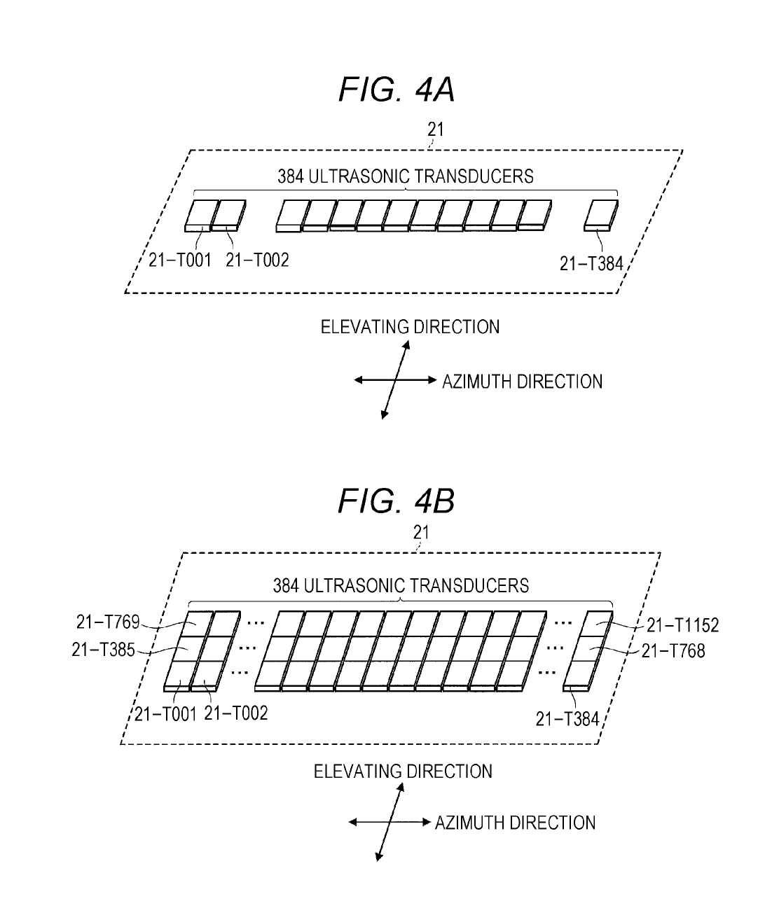

FIGS. 4A and 4B are diagrams showing an example of the array structure of the ultrasound transducers 21. FIG. 4A shows the array structure of the ultrasound transducers 21 according to this embodiment. The 384 ultrasound transducers 21 according to this embodiment are arranged in a row in an azimuth direction in the following order: the ultrasound transducer 21-T001, the ultrasound transducer 21-T002, the ultrasound transducer 21-T003, . . . , and the ultrasound transducer 21-T384.

FIG. 4B shows another form of the array structure of the ultrasound transducers 21. In FIG. 4B, the ultrasound transducers 21 are arranged in an elevating direction as well as the azimuth direction, and 384×3 ultrasound transducers are arranged in the azimuth direction and the elevating direction to form a matrix-like array in a two-dimensional plane. In the array structure of the ultrasound transducers 21 shown in FIG. 4B, 384 ultrasound transducers 21-T001 through 21-T384 are sequentially arranged in the azimuth direction in the first row, 384 ultrasound transducers 21-T385 through 21-T768 are sequentially arranged in the azimuth direction in the second row, and 384 ultrasound transducers 21-T769 through 21-T1152 are sequentially arranged in the azimuth direction in the third row.

Configuration of the Switching Circuits 22

The switching circuits 22 are switching circuits that select ultrasound transducers 21 to be connected to the transmitting/receiving circuit 12 among the ultrasound transducers 21. The switching circuits 22 are typically formed with multiplexer ICs (each of the switching circuits 22-M001 through 22-M192 in FIG. 3 is equivalent to a multiplexer IC, and will be hereinafter also referred to as a “multiplexer 22” or a “MUX 22”). A control line (not shown) to which a control signal from the control unit 11 (the switching control unit 11b) is input is connected to each multiplexer 22, and, in accordance with the control signal, each multiplexer 22 selects a ultrasound transducer 21 to be driven from among the ultrasound transducers 21.

A signal line Lb is connected to the system channel side of each of the 192 multiplexers 22-M001 through 22-M192 according to this embodiment, and signal lines La (two signal lines La in this example) are connected in parallel to each multiplexer on the side of the ultrasound transducers 21. The respective signal lines Lb-001 through Lb-192 are connected to the 192 multiplexers 22-M001 through 22-M192 on the system channel side, and the respective signal lines La-001 through La-384 are connected to the multiplexers 22-M001 through 22-M192 on the side of the ultrasound transducers 21.

Meanwhile, a control signal from the control unit 11 (the switching control unit 11b) is input to each of the multiplexers 22-M001 through 22-M192. In accordance with the control signal, each of the multiplexers 22-M001 through 22-M192, which are independent of one another, selectively connects one of the channels on the side of the ultrasound transducers 21 to one system channel on the side of the transmitting/receiving circuit 12 in an electrical manner. In other words, the 192 multiplexers 22-M001 through 22-M192 are designed to correspond to the system channels Ch-001 through Ch-192, respectively, and independently switch ultrasound transducers 21 to be driven, for each of the system channels.

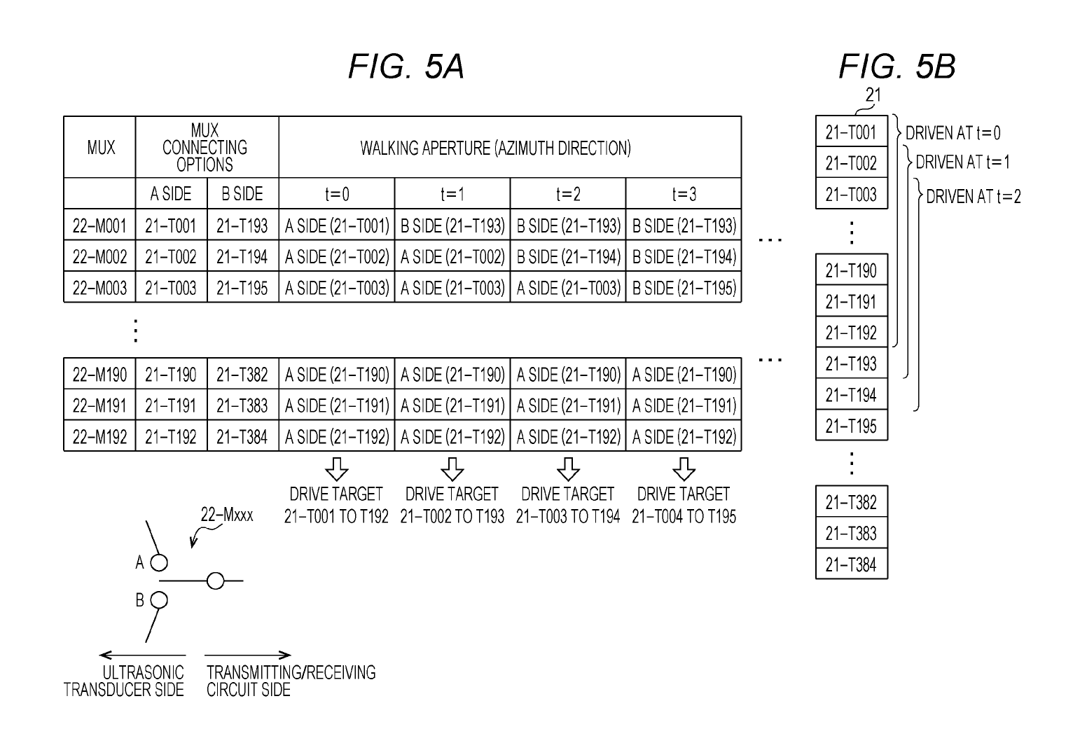

FIGS. 5A and 5B are diagrams for explaining switching operations at the multiplexers 22 according to this embodiment.

FIG. 5A shows electrically connected states of the respective multiplexers 22-M001 through 22-M192 at respective timings in chronological order. FIG. 5B is a diagram schematically showing the ultrasound transducers 21-T001 through 21-T384 to be driven in FIG. 5A.

The left column in FIG. 5A shows the identification numbers of the multiplexers 22-M001 through 22-M192. On the assumption that time elapses in the order of t=0, t=1, t=2, t=3, . . . , the respective rows in FIG. 5A show the electrically connected states of the multiplexers 22-M001 through 22-M192 corresponding to the identification numbers shown in the left column at the respective timings.

In this example, the multiplexer “22-M001” has a signal line La connected to the ultrasound transducer “21-T001” and the ultrasound transducer “21-T193”, and electrically connects one of the two ultrasound transducers to the system channel Ch-001, in accordance with a control signal from the main body 10 (the switching control unit 11b). Likewise, the multiplexer “22-M002” has a signal line La connected to the ultrasound transducer “21-T002” and the ultrasound transducer “21-T194”, and electrically connects one of the two ultrasound transducers to the system channel Ch-002, in accordance with a control signal from the main body 10 (the switching control unit 11b).

The control unit 11 (the switching control unit 11b) determines the ultrasound transducer 21 to be driven among a plurality of ultrasound transducers 21 in a time-sharing manner.

At t=0, the control unit 11 (the switching control unit 11b) sets the electrically connected states of the multiplexers “22-M001” through “22-M192” to A-side connection, so that the ultrasound transducers “21-T001” through “21-T192” are driven.

At t=1, the control unit 11 (the switching control unit 11b) sets the electrically connected state of the multiplexer “22-M001” to B-side connection, and sets the electrically connected states of the multiplexers “22-M002” through “22-M192” to A-side connection, so that the ultrasound transducers “21-T002” through “21-T193” are driven.

At t=2, the control unit 11 (the switching control unit 11b) sets the electrically connected states of the multiplexers “22-M001” and “22-M002” to B-side connection, and sets the electrically connected states of the multiplexers “22-M003” through “22-M192” to A-side connection, so that the ultrasound transducers “21-T003” through “21-T194” are driven.

At t=3, the control unit 11 (the switching control unit 11b) sets the electrically connected states of the multiplexers “22-M001” through “22-M003” to B-side connection, and sets the electrically connected states of the multiplexers “22-M004” through “22-M192” to A-side connection, so that the ultrasound transducers “21-T004” through “21-T195” are driven.

In this manner, the respective multiplexers 22 sequentially select the ultrasound transducers 21 to be connected to the system channels Ch-001 through Ch-192 from among the ultrasound transducers 21-T001 through 21-T384, in accordance with a control signal from the switching control unit 11b. As a result, the scanning block formed with the ultrasound transducers 21 in a driven state sequentially slide with time.

For the respective multiplexers 22-M001 through 22-M192 according to this embodiment, connection targets among the ultrasound transducers 21-T001 through 21-T384 are set, so that the number of connected ultrasound transducers 21 becomes constant (“A-side” or “B-side” in this example) for each one system channel when the scanning block slides. Specifically, of the ultrasound transducers 21-T001 through 21-T384, the same number of adjacent ultrasound transducers 21 as the number of system channels (192 channels in this example) are connected to different multiplexers 22 from each other.

In this manner, the circuit parameters in the circuits between the transmitting/receiving circuit 12 and the ultrasound transducers 21 are kept constant, and the same matching condition for impedance matching in the matching circuit 23 is always maintained.

Configuration of the Matching Circuits 23

The matching circuits 23 are connected to positions directly connected to the multiplexer 22, and perform impedance matching between the multiplexers 22 and the circuits connected to the multiplexers 22. In other words, the matching circuits 23 compensate for the characteristic impedance mismatch caused between circuits due to insertion of the multiplexers 22, and thus, reduces signal degradation.

It should be noted that such a phenomenon of signal reflection often occurs at the boundaries between the multiplexers 22 and the circuits at the subsequent stage of the multiplexers 22, when a transmission signal is transmitted from the transmitting/receiving circuit 12 to the ultrasound transducers 21, or when a reception signal is transmitted from the ultrasound transducers 21 to the transmitting/receiving circuit 12.

In view of this, the matching circuits 23 according to this embodiment are connected to positions connected directly to the multiplexers 22 on the side of the transmitting/receiving circuit 12.

The matching circuits 23 according to this embodiment are also disposed separately from one another in the respective signal lines Lb-001 through Lb-192. In other words, the matching circuits 23 according to this embodiment are formed with the individual matching circuits 23-N001 through 23-N192 connected in series to the subsequent stages of the respective multiplexers 22-M001 through 22-M192.

The matching circuits 23-N001 through 23-N192 typically include inductor elements connected in series, so as to be connected directly to the respective multiplexers 22-M001 through 22-M192.

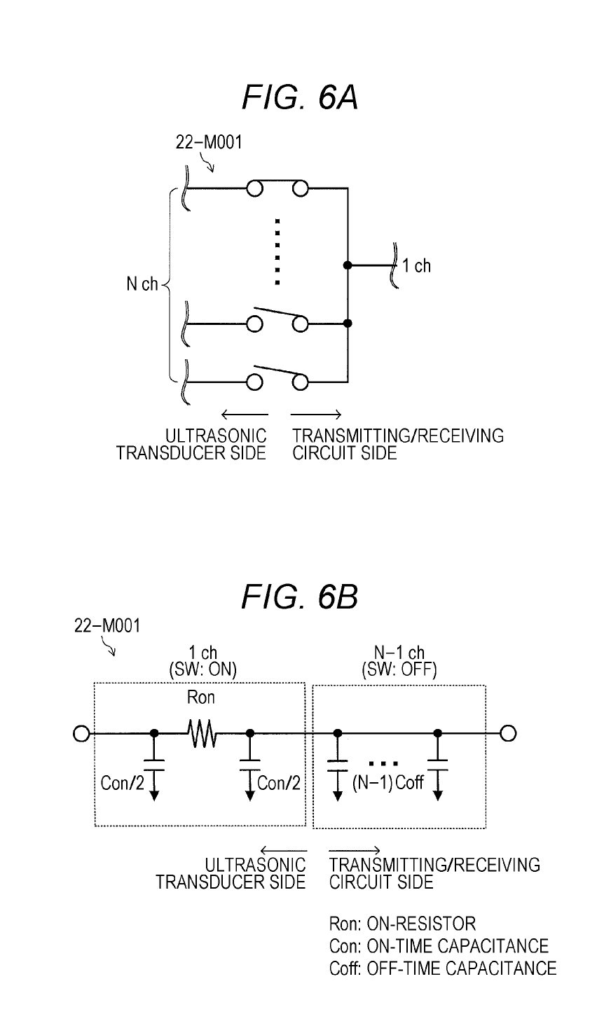

FIGS. 6A and 6B are diagrams showing equivalent circuits of multiplexers 22. Note that the equivalent circuits shown in FIGS. 6A and 6B are known from the following literature, for example: “The Acoustic and Thermal Effects Using Multiplexers in Small Invasive Probes”, L. J. Busse, C. G. Oakley, M. J. Fife, J. V. Ranalletta, R. D. Morgan, and D. R. Dietz, IEEE Ultrasonics Symposium, 1997, 1721-1724.

For one of the system channels, each of the multiplexers 22 according to this embodiment switches on an electrically connected state of one of the N channels (two channels in FIG. 3) of the ultrasound transducers 21 connected thereto via the signal line La. Accordingly, as shown in FIG. 6A, an equivalent circuit of each multiplexer 22 can be expressed as a configuration in which switch portions (the transistors constituting the multiplexer 22, for example) are connected in parallel between the input and the output.

Since each of the multiplexers 22 according to this embodiment is constantly in an electrically connected state on one of “A-side” and “B-side”, and is constantly in an electrically disconnected state on the other side. Accordingly, the impedance formed by the multiplexer 22 always has the same value.

FIG. 6B is an equivalent circuit showing the circuit parameters formed by each multiplexer 22, on the basis of the circuit model shown in FIG. 6A.

As shown in FIG. 6B, the impedance formed by each multiplexer 22 can be expressed as a combination of an on-resistance Ron connected in series between the input and the output in an on-state switch portion, a parasitic capacitance Con/2 connected between the signal line La and the ground in the on-state switch portion, a parasitic capacitance Con/2 connected between the signal line Lb and the ground in the on-state switch portion, and a parasitic capacitance Coff connected between the signal line La and the ground in an off-state switch portion.

The impedance of each multiplexer 22 is normally a value including capacitive components as described above. Further, as the impedance of each multiplexer 22 is a combination of capacitive components connected in parallel, the capacitive components easily become larger, and characteristic impedance mismatch is often caused, particularly at the boundaries with the signal lines connected to the multiplexers 22 on the system channel side.

In view of this, the circuit constant of each matching circuit 23 is set so that the reactance component that is the sum of the impedances of the multiplexer 22 and the matching circuit 23 becomes zero, for example. For example, the circuit constant of each matching circuit 23 is set so that the resistive component that is the sum of the impedances of the multiplexer 22 and the matching circuit 23 becomes close to the characteristic impedance (50Ω, for example) of the signal line Lb connected to the matching circuit 23.

Alternatively, the circuit constant of each matching circuit 23 may be set so that the electrical impedance of the ultrasound probe 20 is electrically matched with the input/output impedance of the transmitting/receiving unit 12, for example. In that case, however, while the output impedance of the transmitting portion of the transmitting/receiving unit 12 is several tens of Ω, the input impedance of the receiving portion is several hundreds of Ω. Therefore, the circuit constant of each matching circuit 23 may be set so that electrical matching can be achieved between the transmitting portion and the receiving portion.

In the above manner, reflection phenomena at the boundaries between the multiplexers 22 and the circuits on the subsequent-stage side of the multiplexers 22 can be reduced, both when a transmission signal is transmitted from the transmitting/receiving circuit 12 to the ultrasound transducers 21, and when a reception signal is transmitted from the ultrasound transducers 21 to the transmitting/receiving circuit 12.

A configuration having an inductance value is typically selected as the configuration of each matching circuit 23, because the impedance of each multiplexer 22 is inclined toward the capacitive component side. The configuration of each matching circuit 23 typically includes inductor elements that are connected in series, as described above.

However, the matching circuits 23-N001 through 23-N192 do not necessarily include inductor elements connected in series, and may of course have a configuration that performs impedance matching depending on the line length of a transmission line (λ/4 line, for example), or a configuration that performs impedance matching using stubs or the like.

It is also possible to set the circuit constant of each matching circuit 23, taking into account not only the impedance of the multiplexer 22, but also the impedance of the signal line La on the side of the ultrasound transducer 21, the impedance of the ultrasound transducer 21, the impedance of the signal line Lb on the side of the transmitting/receiving circuit 12 (including the area leading to the transmission/reception circuit 12), and the like. In a case where the condition for impedance matching differs between transmission of a transmission signal from the transmitting/receiving circuit 12 to the ultrasound transducers 21 and transmission of a reception signal from the ultrasound transducers 21 to the transmitting/receiving circuit 12, on the other hand, the circuit constant of each matching circuit 23 may be set depending on the case where reflection phenomena are to be reduced.

In such a case, the circuit constant of each matching circuit 23 can be designed by the same technique as a known technique for impedance matching. For example, it is possible to use a technique for setting a circuit constant so that the impedance on the side of the transmitting/receiving circuit 12 has a complex conjugate relationship with the impedance on the side of the ultrasound transducer 21, with the reference point being the connecting point on the subsequent-stage side of the matching circuit 23, and the impedance on the side of the transmitting/receiving circuit 12 has a complex conjugate relationship with the impedance on the side of the ultrasound transducer 21, with the reference point being the connecting point on the preceding-stage side of the matching circuit 23.

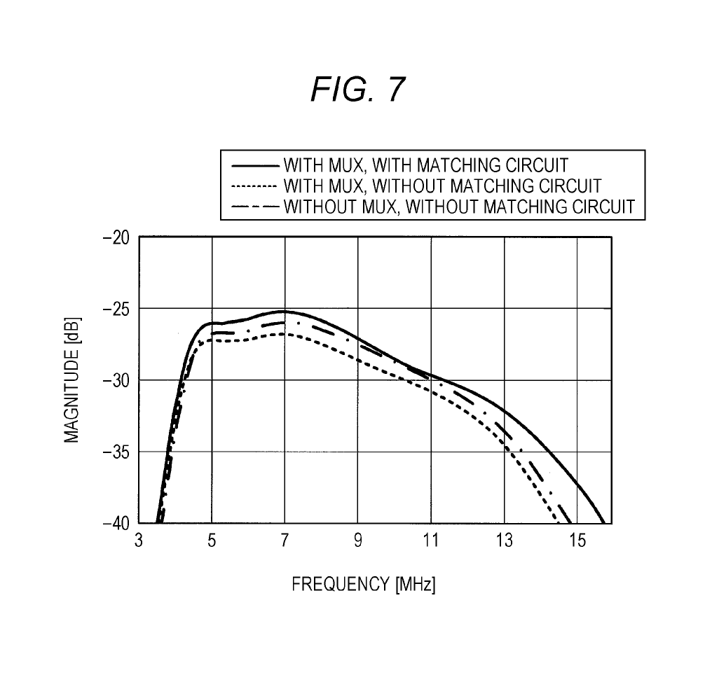

FIG. 7 shows the results of a simulation conducted to determine the transmission/reception characteristics of the ultrasound probe 20 according to this embodiment.

In this simulation, predetermined circuit parameters are set for the respective components in the circuit configuration of the ultrasound probe 20 shown in FIG. 3, and transmission/reception characteristics are calculated. In this simulation, transmission/reception characteristics are calculated in a series of processes: a transmission signal is transmitted from the transmitting/receiving circuit 12 to the ultrasound transducers 21, the ultrasound transducers 21 are made to transmit ultrasound waves, the ultrasound echo returned from a predetermined target is converted into a reception signal at the ultrasound transducers 21, and the reception signal returned to the transmitting/receiving circuit 12 is acquired.

The transmission/reception characteristics calculated in this simulation indicate the degree of decrease in the signal strength of the reception signal returned to the transmitting/receiving circuit 12 with respect to the signal strength of the transmission signal transmitted from the transmitting/receiving circuit 12 to the ultrasound transducers 21. In this simulation, a degree of decrease in signal strength is also calculated at each frequency of transmission signals.

In the graph shown in FIG. 7, the solid line indicates the transmission/reception characteristics in the circuit configuration shown in FIG. 3 (which includes the multiplexers 22 and the matching circuits 23). The dot-and-dash line indicates the transmission/reception characteristics in a mode that has the circuit configuration shown in FIG. 3 but do not include the multiplexers 22 and the matching circuits 23. The dotted line indicates the transmission/reception characteristics in a mode that has the circuit configuration shown in FIG. 3 but do not include the matching circuits 23.

In FIG. 7, the abscissa axis indicates transmission signal frequency, and the ordinate axis indicates the degree of decrease in signal strength when the ultrasound transducers 21 perform transmission and reception (=the ratio of the power of the transmission signal transmitted from the transmitting/receiving circuit 12 to the power of the reception signal acquired by the transmitting/receiving circuit 12). The circuit constants of the respective components shown in FIG. 3 are the same in the respective modes.

As can be seen from FIG. 7, in the case where the multiplexers 22 are included (the dotted line), the decrease in signal strength is smaller than that in the mode without the multiplexers 22 and the matching circuits 23 (the dot-and-dash line). In the case where the matching circuits 23 are included (the solid line) as in the ultrasound probe 20 according to this embodiment, the degree of decrease in signal strength is smaller than that in the case where the matching circuits 23 are not included (the dotted line).

Furthermore, in the ultrasound probe 20 according to this embodiment (the solid line), the degree of decrease in signal strength is substantially the same as that in the mode without the multiplexers 22 and the matching circuits 23 (the dot-and-dash line). This proves that the matching circuits 23 can substantially compensate for signal degradation caused by the multiplexers 22.

[Mounting Structure for the Ultrasound Probe]

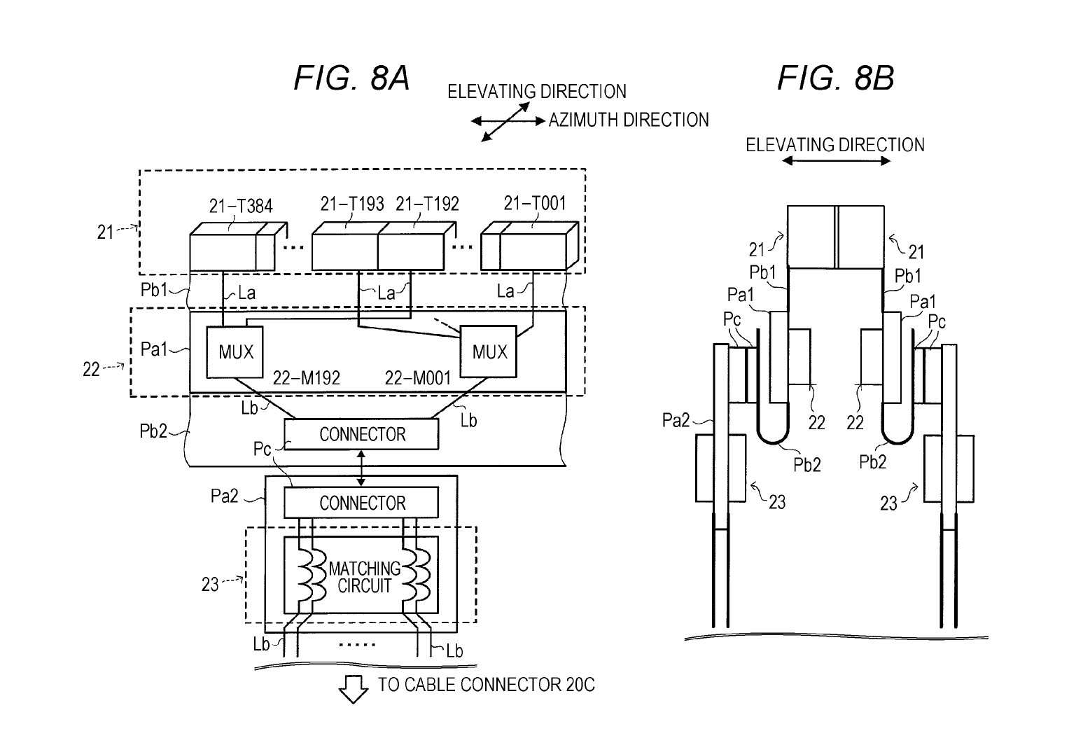

FIGS. 8A and 8B are diagrams showing a mounting structure for the circuit components of the ultrasound probe 20 according to this embodiment. FIG. 8A is a plan view of the mounting structure, and FIG. 8B is a side view of the mounting structure. It should be noted that, in FIG. 8B, a pair of mounting structures, each of which is the same as the mounting structure shown in FIG. 8A, are mounted.

The ultrasound probe 20 according to this embodiment includes a first circuit board Pa1 in which the multiplexers 22 are mounted, a second circuit board Pa2 in which the matching circuits 23 are mounted, a first flexible wiring board Pb1 that connects the ultrasound transducers 21 and the first circuit board Pa1 with wires, and a second flexible wiring board Pb2 that connects the first circuit board Pa1 and the second circuit board Pa2 with wires.

In this mode, the switching circuits 22 are formed with individual multiplexer ICs. A multilayer wiring board is used as the first circuit board Pa1 in which the multiplexers 22 are mounted. With this arrangement, the signal lines La-001 through La-384 corresponding to the large number of channels of the ultrasound transducers 21-T001 through 21-T384 can be formed in the substrate of the first circuit board Pa1.

In each of the first flexible wiring board Pb1 and the second flexible wiring board Pb2, a wiring part is formed with an anisotropic conductive film, for example. A connector-connector connection Pc is used as the connecting structure between the wiring part of the second circuit board Pa2 and the wiring part of the second flexible wiring board Pb2.

As described above, in the ultrasound probe 20 according to this embodiment, the multiplexers 22 and the matching circuits 23 are mounted in the different circuit boards Pa1 and Pa2, and the circuit boards Pa1 and Pa2 are connected by the first flexible wiring board Pb1. With this arrangement, an increase in the size of the circuit boards is prevented, and the ultrasound probe 20 is made smaller in size. In other words, this structure increases the degree of freedom in the layout of the components in the ultrasound probe 20. Thus, it is possible to insert the matching circuits 23 without affecting the exterior of the ultrasound probe 20.

To achieve a smaller size, two mounting structures, each of which is the same as that shown in FIG. 8A, are preferably mounted as a pair in the ultrasound probe 20, as shown in FIG. 8B. Specifically, two mounting structures, each of which is the same as that shown in FIG. 8A, are preferably provided as a pair so that the respective board surfaces of the first circuit board Pa1 and the second circuit board Pa2 face each other. With this arrangement, it is possible to form the signal lines La-001 through La-384 corresponding to the large number of channels of the ultrasound transducers 21-T001 through 21-T384.

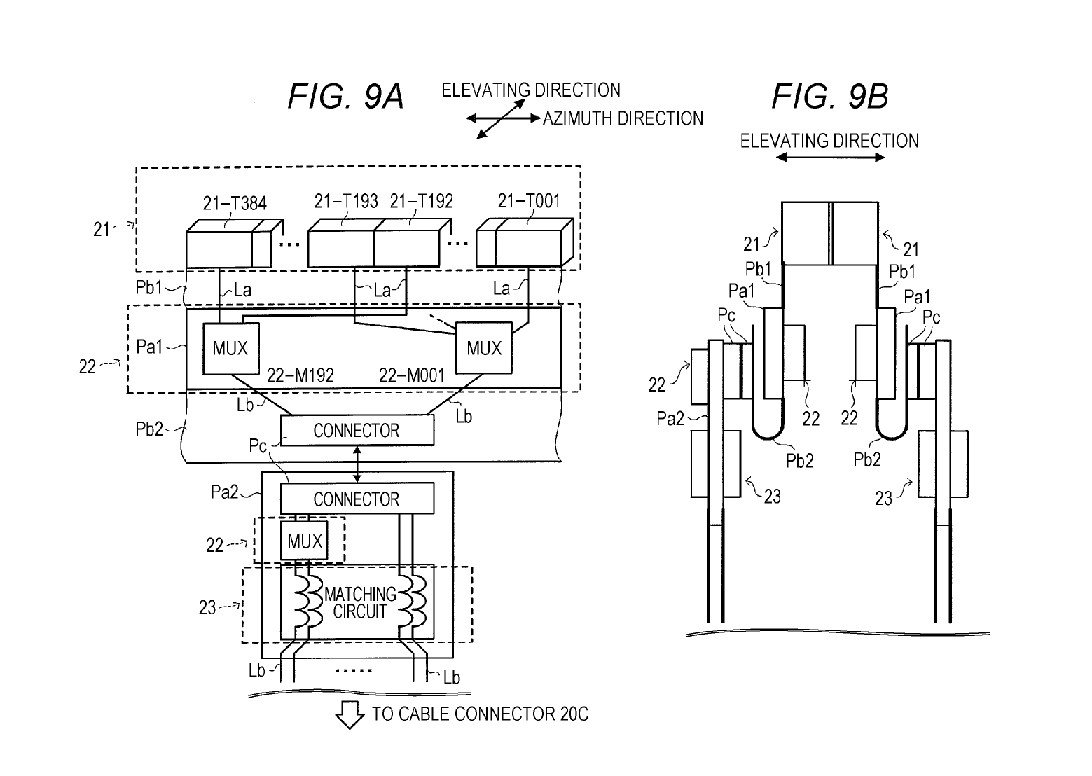

FIGS. 9A and 9B show a modification of the mounting structure shown in FIGS. 8A and 8B. FIG. 9A is a plan view of a mounting structure according to this modification, and FIG. 9B is a side view of the mounting structure according to this modification.

The mounting structure shown in FIGS. 9A and 9B differs from the mounting structure shown in FIGS. 8A and 8B only in that some of the multiplexers 22 are mounted in the first circuit board Pa1, and the other multiplexers 22 are mounted in the second circuit board Pa2. As the multiplexers 22 are disposed on both the first circuit board Pa1 and the second circuit board Pa2, an increase in the size of the first circuit board Pa1 can be more effectively prevented.

[Effects]

As described above, in the ultrasound probe 20 of the ultrasound diagnostic apparatus 1 according to this embodiment, the matching circuits 23 that perform impedance matching between the multiplexers 22 (equivalent to switching circuits) and the circuits on the subsequent-stage side connected to the multiplexers 22 are connected to the positions connected directly to the multiplexers 22 on the side of the transmitting/receiving circuit 12.

With this configuration, reflection phenomena at the connecting positions between the multiplexers 22 and the circuits in the subsequent stage can be reduced, when a transmission signal is transmitted from the transmitting/receiving circuit 12 to the ultrasound transducers 21, or when a reception signal is transmitted from the ultrasound transducers 21 to the transmitting/receiving circuit 12. In other words, this configuration can reduce signal degradation due to reflection phenomena, and improve the acoustic characteristics of the ultrasound transducers 21.

Further, in the ultrasound probe 20 according to this embodiment, it is possible to overcome the difficulty in reducing the circuit size while providing the matching circuits 23 between the multiplexers 22 and the respective ultrasound transducers 21 so as to perform impedance matching at the positions of connection to the subsequent stage of the multiplexers 22.

Furthermore, in the ultrasound probe 20 according to this embodiment, the number of ultrasound transducers 21 to be connected to the transmitting/receiving circuit 12 by the multiplexers 22 is maintained constant at each timing during electronic scanning. Thus, the same matching condition for impedance matching at the matching circuits 23 can be always maintained.

### Second Embodiment

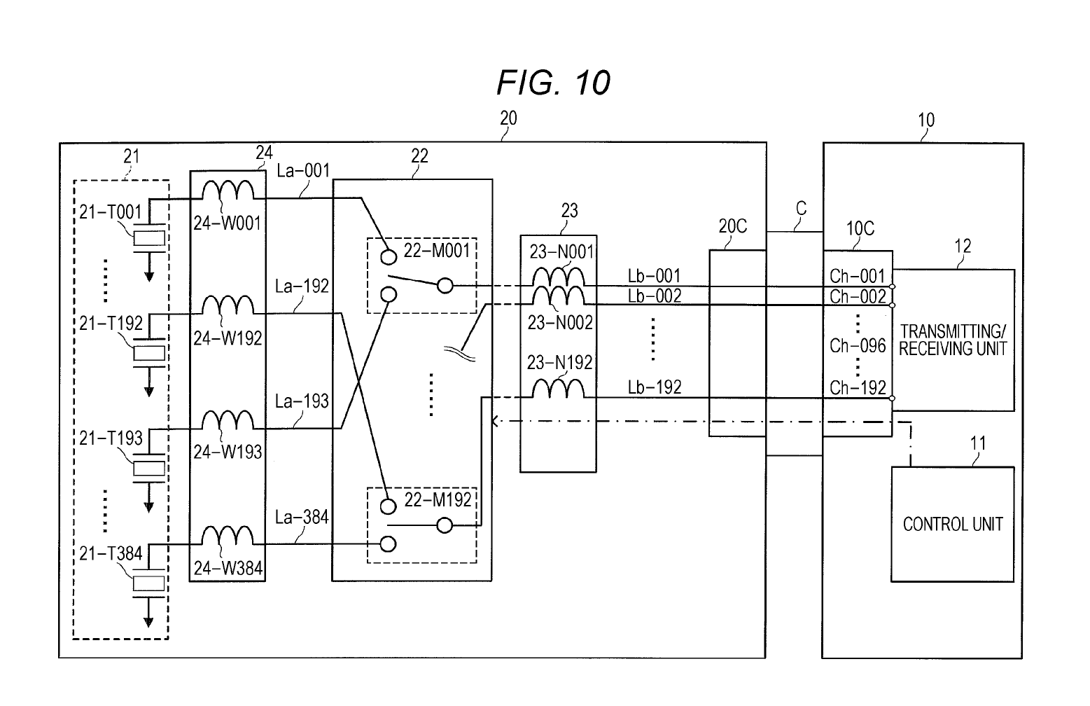

Next, an example of the configuration of an ultrasound probe 20 according to a second embodiment is described, with reference to FIG. 10.

FIG. 10 is a circuit diagram showing the configuration of the ultrasound probe 20 according to this embodiment. The ultrasound probe 20 according to this embodiment differs from the ultrasound probe 20 according to the first embodiment in further including matching circuits 24 on the preceding-stage side of positions connected directly to the multiplexers 22. For clear distinction, the matching circuits 23 on the subsequent-stage side of the multiplexers 22 will be hereinafter referred to as the “first matching circuits 23”, and the matching circuits 24 on the preceding-stage side of the multiplexers 22 will be hereinafter referred to as the “second matching circuits 24”.

The second matching circuits 24 are connected in series to the respective ultrasound transducers 21-T001 through 21-T384 on the preceding-stage side of the multiplexers 22-M001 through 22-M192. That is, the ultrasound probe 20 according to this embodiment includes 384 second matching circuits 24-W001 through 24-W384.

Like the first matching circuits 23, the second matching circuits 24 are typically formed with series-connected inductor elements or the like.

As described above, in the ultrasound probe 20 of the ultrasound diagnostic apparatus 1 according to this embodiment, the second matching circuits 24 that perform impedance matching between the multiplexers 22 (equivalent to switching circuits) and the circuits on the preceding-stage side connected to the multiplexers 22 are connected to the positions connected directly to the multiplexers 22 on the side of the ultrasound transducers 21. With this configuration, reflection phenomena can be even more effectively reduced.

### Other Embodiments

The present invention is not limited to the above embodiments, and various modifications can be made to them.

In the above embodiments, as an example of the switching circuits 22, the individual multiplexers 22-M001 through 22-M192 are provided for the respective system channels Ch-001 through Ch-192 of the transmitting/receiving circuit 12. However, the switching circuit 22 may be formed with any structures that are capable of selectively connecting the ultrasound transducers 21 to be driven to the transmitting/receiving circuit 12.

Further, in an example of the ultrasound probe 20 in the above embodiments, the ultrasound transducers 21 have the same structures, the switching circuits 22 have the same structures, and the matching circuits 23 have the same structures. However, some of these components may of course be different.

Also, in an example of the ultrasound probe 20 in the above embodiments, all the transmitting circuits that transmit transmission signals to the ultrasound transducers 21, and all the receiving circuits that acquire reception signals from the ultrasound transducers 21 and perform a reception process on the reception signals are disposed in the main body 10 of the ultrasound diagnostic apparatus 1. However, some or all of these components may be disposed in the housing of the ultrasound probe 20.

With an ultrasound probe according to an embodiment of the present disclosure, it is possible to reduce degradation of acoustic characteristics due to switching circuits that selectively switch ultrasound transducers to be driven among a plurality of ultrasound transducers.

Although embodiments of the present invention have been described and illustrated in detail, the disclosed embodiments are made for purposes of illustration and example only and not limitation. The scope of the present invention should be interpreted by terms of the appended claims. The inventions disclosed in the claims include technologies achieved by making various changes and modifications to the above specific embodiments.

What is claimed is:

1. An ultrasound probe of an ultrasound diagnostic apparatus, comprising:
a plurality of ultrasound transducers that perform mutual conversion between an ultrasound wave and an electrical signal;
a switching circuit that selectively switches ultrasound transducers to be electrically connected to a transmitting/receiving circuit among the plurality of ultrasound transducers; and
a matching circuit that is provided at one of a preceding stage and a subsequent stage of the switching circuit, is connected to a position connected directly to the switching circuit, and performs impedance matching between the switching circuit and a circuit disposed at one of the preceding stage and the subsequent stage of the switching circuit.

2. The ultrasound probe according to claim 1, wherein, on a subsequent-stage side of the switching circuit, the matching circuit is connected to a position connected directly to the switching circuit.

3. The ultrasound probe according to claim 1, wherein the matching circuit is connected to both the preceding stage and the subsequent stage of the switching circuit.

4. The ultrasound probe according to claim 1, wherein the switching circuit is a plurality of multiplexers corresponding to a plurality of system channels, respectively, the transmitting/receiving circuit having the plurality of system channels.

5. The ultrasound probe according to claim 4, wherein, on a subsequent-stage side of the switching circuit, the matching circuit is provided for each signal line of the plurality of system channels so as to be connected to each of the plurality of multiplexers.

6. The ultrasound probe according to claim 5, wherein the matching circuit includes a series-connected inductor element for each signal line of the plurality of system channels.

7. The ultrasound probe according to claim 5, wherein a circuit constant of the matching circuit is set so as to make a reactance component of the multiplexers zero.

8. The ultrasound probe according to claim 4, wherein the number of the ultrasound transducers to be electrically connected to the transmitting/receiving circuit by the multiplexers is maintained constant at each timing during electronic scanning.

9. The ultrasound probe according to claim 1, wherein the transmitting/receiving circuit is disposed in a main body of the ultrasound diagnostic apparatus.

10. The ultrasound probe according to claim 1, wherein
the switching circuit is mounted in a first circuit board,
the matching circuit is mounted in a second circuit board, and
a wiring part of the first circuit board and a wiring part of the second circuit board are connected via a flexible wiring board.

11. The ultrasound probe according to claim 10, wherein a wiring part of the flexible wiring board includes an anisotropic conductive film.

12. An ultrasound diagnostic apparatus comprising the ultrasound probe according to claim 1.
